(19) United States
(12) Patent Application Publication
(10) Pub. No.: US 2026/0266751 A1
(43) Pub. Date: Sep. 10, 2026

(54) SOIL CORROSIVITY SIMULATOR

(71) Applicant: Saudi Arabian Oil Company, Dhahran (SA)

(72) Inventors: Turki Abdullah Al-Khaldi, Dammam (SA); Mozah M. Almulhim, Dammam (SA); Naim Dakwar, Dhahran (SA); Zahrah S. Altarooti, Tarout (SA)

(21) Appl. No.: 19/072,036

(22) Filed: Mar. 6, 2025

(51) Int. Cl. G01N 27/04; G01N 33/24

(52) U.S. Cl. CPC G01N 27/043; G01N 33/24

(57) ABSTRACT

Systems and methods include a simulation process of soil corrosion. The simulation environment of soil corrosion includes an autoclave chamber for temperature, humidity, and pressure control of a soil sample. The corrosion rate is measured utilizing a four-way electrical resistance probe inserted at different locations in the soil sample. The properties of the soil are assessed utilizing a resistivity testing box and a potential of hydrogen (pH) sensor or oxidation reduction potential (ORP) probe inserted in the soil sample. The effect of packing density of the soil on the corrosion rate is assessed by incorporating a force exertion system. The corrosion rate is also measured for different moisture content levels. A potential to minimize the soil corrosion rate in the region of interest to protect the buried pipelines can be adjusted.

### TECHNICAL FIELD

The present disclosure is generally related to pipeline operation and, more specifically, to soil corrosion protection.

### BACKGROUND

Pipelines in the oil and gas industry are often made from materials like steel, which is selected for the characteristic strength and durability, characteristics that are essential for withstanding the high pressures and harsh conditions in oil and gas transportation. A large section of oil and gas pipelines are buried in soil for minimizing impact from physical damage caused by weather, human activities, and natural disasters. The subterranean region forms a controlled environment but is susceptible to corrosion. Soil corrosion can lead to asset exposure that can eventually lead to leaks and spills, posing environmental hazards and safety risks. Effective soil corrosion management strategies are crucial to maintaining the integrity of buried assets and ensuring safe and reliable oil and gas operations.

### SUMMARY

Implementations of the present disclosure are directed to pipeline operation. More particularly, implementations of the present disclosure are directed to soil corrosivity modelling for soil corrosion protection.

In some implementations, a method includes: setting a temperature inside an autoclave chamber to simulate corrosion conditions of a soil sample extracted from a region of interest including buried pipelines, adjusting a moisture level inside the autoclave chamber to simulate moisture corrosion conditions, activating a force exertion system to apply a controlled pressure onto the soil sample at the moisture level, receiving, from a four-way electrical resistance probe, electrical resistance data of the soil, determining a soil corrosivity by processing the electrical resistance data, the soil corrosivity defining a soil corrosion rate of the soil sample relative to the temperature and the moisture level at the controlled pressure, and adjusting a potential to minimize the soil corrosion rate in the region of interest to protect the buried pipelines.

The foregoing and other implementations can each optionally include one or more of the following features, alone or in combination. In particular, implementations can include all the following features:

In a first aspect, combinable with any of the previous aspects, the four-way electrical resistance probe includes four separate electrodes, a potentiostat, and an electrical resistance data logger, wherein the four separate electrodes are placed in a substantially straight line on a surface of the soil. In another aspect, combinable with any of the previous aspects, the computer-implemented method further includes adjusting a polarization of the potentiostat connected to the electrical resistance data logger for potential control. In another aspect, combinable with any of the previous aspects, determining the soil corrosivity includes measuring a metal loss and adjusting the potential to minimize the soil corrosion includes an adjustment of a cathodic protection. In another aspect, combinable with any of the previous aspects, the computer-implemented method further includes receiving from a pH probe inserted into or contacting the soil, an acidity level of the soil, wherein determining soil corrosivity includes processing the electrical resistance data and the acidity level of the soil. In another aspect, combinable with any of the previous aspects, the computer-implemented method further includes triggering an introduction of corrosive substances including hydrogen sulfide, and carbon dioxide to replicate conditions causing the soil corrosivity.

Other implementations of the aspect include corresponding systems, apparatus, and computer programs, configured to perform the actions of the methods, encoded on computer storage devices.

The present disclosure also provides a computer-readable storage medium coupled to one or more processors and having instructions stored thereon which, when executed by the one or more processors, cause the one or more processors to perform operations in accordance with implementations of the methods provided herein.

The present disclosure further provides a system for implementing the methods provided herein. The system includes one or more processors, and a computer-readable storage medium coupled to the one or more processors having instructions stored thereon which, when executed by the one or more processors, cause the one or more processors to perform operations in accordance with implementations of the methods provided herein.

It is appreciated that methods in accordance with the present disclosure can include any combination of the aspects and features described herein. That is, methods in accordance with the present disclosure are not limited to the combinations of aspects and features described herein, but also include any combination of the aspects and features provided.

Implementations described in the present disclosure, provide an accurate identification of impact of soil properties on soil corrosion rate, facilitating application of corrosion protection mechanisms to ensure the reliable, safe, and continuous oil and gas operations. The described approach provides efficient extraction and analysis of corrosion rate relative to the properties of the soil. The proposed design provides a comprehensive testing apparatus that improves the simulation process of soil corrosion. Another advantage of the described technology is that the described corrosion rate is measured utilizing a four-way electrical resistance probe at different locations in the system that increases the accuracy of the results. The described technology substantially improves over existing methods in that it integrates multiple soil properties (electrical resistance, acidity level, packing density, and moisture level) to identify corrosion rate relative to changing soil properties. The timely identification of corrosion rate relative to changing soil properties advantageously facilitates activation of effective preventative measures to avoid and/or correct cable corrosion within separate soil sections. The preventative measures include cathodic protection control by automatically adjusting the electrical current supplied to pipelines to prevent corrosion, using real-time data from soil corrosivity sensors to maintain optimal protection levels. Another automatic corrosion prevention operation can include application of advanced materials and coatings that respond to changes in soil corrosivity to self-heal and change properties to resist corrosion, enhancing the longevity of infrastructure and system safety.

The details of one or more implementations of the subject matter of the specification are set forth in the accompanying drawings and the description below. Other features, aspects, and advantages of the subject matter can become apparent from the description, the drawings, and the claims.

### DESCRIPTION OF THE DRAWINGS

The accompanying drawings, which are incorporated in and constitute a part of this specification, show particular aspects of the subject matter disclosed herein and, together with the description, help explain some of the principles associated with the disclosed implementations. In the drawings,

FIG. 1A is a block diagram of an example system that can be used to execute implementations of the present disclosure.

FIG. 1B is a block diagram of a portion of the example system that can be used to execute implementations of the present disclosure.

FIG. 2 illustrates an example soil corrosion protection system, according to some implementations of the present disclosure.

FIG. 3 is a flowchart illustrating an example process for cable exposure identification, in accordance with some example embodiments.

FIG. 4 depicts a block diagram illustrating a computing system, in accordance with some example embodiments.

When practical, like labels are used to refer to same or similar items in the drawings.

### DETAILED DESCRIPTION

The following detailed description describes techniques for soil corrosion identification. More particularly, implementations of the present disclosure are directed to soil corrosion simulation relative to soil properties. The described implementations provide methods and systems for determining impact of soil properties on soil corrosion using a simulation process of soil corrosion. The simulation environment of soil corrosion includes an autoclave chamber for temperature and pressure control of a soil sample. The corrosion rate is measured utilizing a four-way electrical resistance probe inserted at different locations in the soil sample. The properties of the soil are assessed utilizing a resistivity testing box and a potential of hydrogen (pH) sensor or oxidation reduction potential (ORP) probe inserted in the soil sample. The effect of packing density of the soil on the corrosion rate is assessed by incorporating a force exertion system. The corrosion rate is also measured for different moisture content levels.

Soil properties can have a significant impact on soil corrosion. Soil properties that can affect soil corrosion include chemical composition, temperature, pressure, pH levels, moisture level, and packing density. Soil properties are unevenly distributed in the three-dimensional space and display diverse patterns that change over time with weather events and other parameters. Comprehensive mapping of soil properties relative to environmental factors can facilitate monitoring and prevention of soil corrosion for safe pipeline operations.

Some traditional studies of soil corrosion have utilized pipeline surveys that focus only on measuring and evaluating the soil corrosion rate without assessing the properties of the soil. However, analysis of soil corrosion rate independent of associated soil properties provide results limited to particular subterranean regions and time intervals. Given that oil and gas pipelines are installed over vast distances, robust soil corrosion simulations that integrate soil property variations can ensures adequate and reliable monitoring of soil corrosion to facilitate continuous and safe pipeline operations.

The techniques described in the present disclosure provide a soil corrosion simulation technique, addressing the challenge of traditional techniques in soil corrosion that fail to consider soil properties. The described approach is based on integration of soil properties in soil corrosion simulations that minimizes errors related to soil property changes that affected to traditional methods. Soil samples can be collected from various sites surrounding operating pipelines. The described approach includes a variation of multiple soil properties to facilitate a comprehensive estimation of their impact on soil corrosion. The proposed approach measures corrosion rate utilizing a four-way electrical resistance probe. The timely identification of corrosion rate relative to changing soil properties advantageously facilitates activation of effective preventative measures to avoid and/or correct cable corrosion within multiple soil regions having different soil properties. Another advantage of the described technology is that the automatic implementation of preventative measures by triggering of automatic operations for systems and machines configured to maintain reliable, safe, and continuous oil and gas operations. The preventative measures can include cathodic protection control by automatically adjusting the electrical current supplied to portions of pipelines to prevent corrosion, using sample-based data from respective soil corrosivity tests to maintain optimal protection levels along pipelines. Another automatic corrosion prevention operation can include application of advanced materials and coatings that respond to changes in soil corrosivity to self-heal and change properties to resist corrosion along pipelines, enhancing the integrity of pipelines and safe leak free operations.

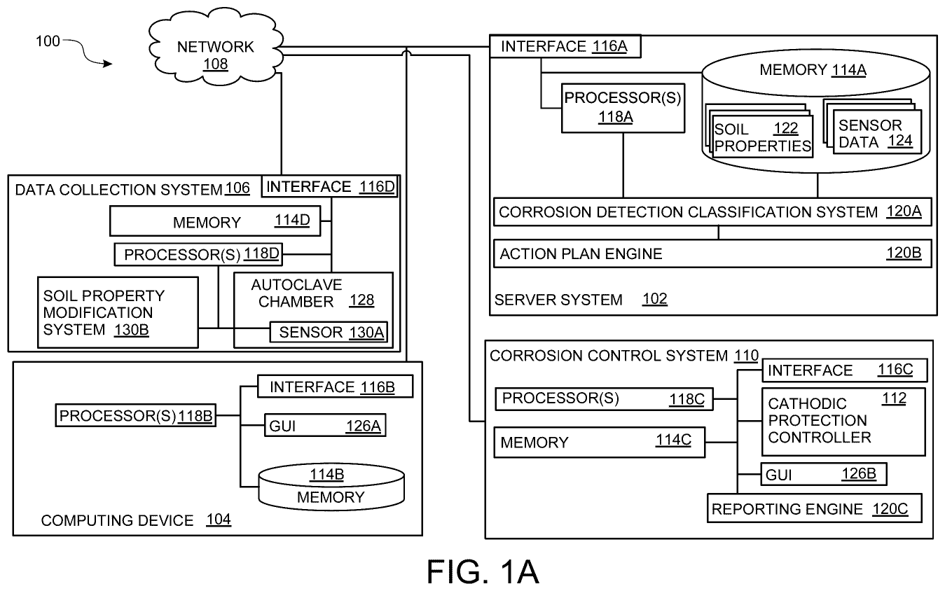

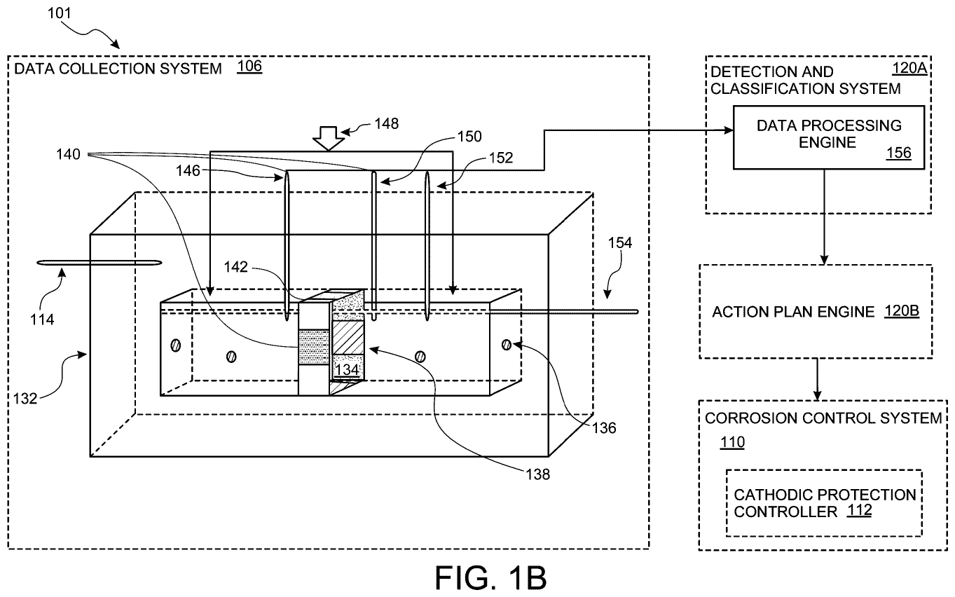

FIG. 1A is a block diagram illustrating an example system 100 that can be used to execute implementations of the present disclosure. For example, example system 100 can be configured to execute simulation of soil corrosion. The illustrated example system 100 includes or is communicably coupled with a server system 102, a computing device 104, a data collection system 106, a network 108, and a corrosion control system 110. Although shown separately, in some implementations, functionality of two or more systems or components of the example system 100 may be provided by a single system or server. In some implementations, the functionality of one illustrated system, server, or component may be provided by multiple systems, servers, or components, respectively.

In the example of FIG. 1A, the server system 102 is intended to represent various forms of servers including, but not limited to a web server, an application server, a proxy server, a network server, and/or a server pool. In general, the server system 102 manages clustering algorithms for unsupervised segmentation of soil properties. In accordance with implementations of the present disclosure, and as noted above, the server system 102 can host a solution environment that can be a cloud environment providing software applications, systems, and services that can be consumed by customers as a service. In some implementations, the server system 102 can support configuring of various data collection systems 106, as well as different types of corrosion control systems 110.

For example, the server system 102 includes a memory 114A, an interface 116A, a processor 118A, and a corrosion detection and classification system 120A and an action plan engine 120B. The memory 114A can include soil properties 122 and sensor data 124. The soil properties 122 include data measured by and received from the data collection system 106. The soil properties 122 can include parameters set by the data collection system 106. The sensor data 124 can be detected by one or more sensors 130A of the data collection system 106. The soil properties 122 and the sensor data 124 can be processed by the corrosion detection and classification system 120A to generate soil corrosion estimates that are processed by the action plan engine 120B to generate action plans defining remedial operations performed by the corrosion control system 110.

The computing device 104 and the corrosion control system 110 may each include any computing device operable to connect to or communicate in the network(s) 108 using a wireline or wireless connection. In general, each of the computing device 104 and the corrosion control system 110 includes an electronic computer device operable to receive, transmit, process, and store any appropriate data associated with the example system 100 of FIG. 1A. Each of the computing device 104 and the corrosion control system 110 can encompass any client computing device such as a laptop/notebook computer, wireless data port, smart phone, personal data assistant (PDA), tablet computing device, one or more processors within these devices, or any other suitable processing device. The computing device 104, the data collection system 106, and the corrosion control system 110, respectively include interface(s) 116B, 116C, 116D, processor(s) 118B, 118C, 118D, and memories 114B, 114C, 114D.

The computing device 104 can include a graphical user interface (GUIs) 126A. For example, the GUI 126A includes an input device, such as a keypad, touch screen, or other device that can accept user information, and an output device that conveys information associated with the operation of the server system 102, or the client device itself, including a display of the material maps and action plan operations selected based on the material movement patterns. The GUI 126A can interface with at least a portion of the example system 100 for any suitable purpose, including generating a visual representation of the soil corrosion relative to soil properties, as collected by the data collection system 106, the material maps generated by the server system 102, or data stored by the server system 102, such as soil properties 122 and sensor data 124, respectively. In particular, the GUI 126A may each be used to view and adjust various sensor data. Generally, the GUI 126A provides the user with an efficient and user-friendly presentation of the soil corrosion relative to soil properties communicated within the example system 100. The GUI 126A may each include multiple customizable frames or views having interactive fields, for selection of regions of interest and/or display of maps of soil properties for different regions and time points. The GUI 126A can be any suitable graphical user interface, such as a combination of a generic web browser, intelligent engine, and command line interface (CLI) that processes information and efficiently presents the results to the user visually.

The corrosion control system 110 can include a reporting engine 120C, a cathodic protection controller 112, the GUI 126B (dashboard), an interface 116C, and a processor 118C. The reporting engine 120C utilizes the analytics data provided by the action plan engine 120B to generate triggers that activate the cathodic protection controller 112. The reporting engine 120C can provide support for soil corrosion control actions in addition to basic (preset standard) corrosion control actions for pipeline integrity and continuous operability. The reporting engine 120C can facilitate modification of operations of the cathodic protection controller 112 and other corrosion control systems and machines selected for corrosion control throughout different soil conditions along pipelines.

The data collection system 106 can include multiple sensors 130A attached to the autoclave chamber 128 or inserted in a soil sample of the autoclave chamber 128 and a soil property modification system 130B. The sensors 130A can include a four-way electrical resistance probe, a pH sensor, an ORP probe, a thermometer, a pressure sensor, and other sensors that can measure soil properties. The soil property modification system 130B can include a force exertion system, a sprinkler system to modify soil sample moisture level, a thermostat, a heating and cooling system to modify a temperature of the autoclave chamber 128, and a corrosive substance supply system to inject corrosive substances into the soil sample of the autoclave chamber 128. The processor 118D of the data collection system 106 controls operation of the sensors 130A and the soil property modification system 130B. The sensors 130A can collect soil properties of the soil sample analyzed within the autoclave chamber 128, while the soil property modification system 130B modifies one or more properties of the soil sample. Further details about the sensors 130A and the soil property modification system 130B and their operation are provided with reference to FIG. 1B.

In some implementations, the network 108 can include a large computer network, such as a local area network, a wide area network, the Internet, a cellular network, a telephone network, or any appropriate combination thereof connecting any number of communication devices, mobile computing devices, fixed computing devices and server systems. Data exchanged over the network 108, is transferred using any number of network layer protocols, such as Internet Protocol, Multiprotocol Label Switching, Asynchronous Transfer Mode, Frame Relay, etc. Furthermore, in implementations where the network 108 represents a combination of multiple sub-networks, different network layer protocols are used at each of the underlying sub-networks. In some implementations, the network 108 represents one or more interconnected internetworks, such as the public Internet.

Each processor 118A, 118B, 118C, 118D included in different components of the example system 100 can include a central processing unit, an application particular integrated circuit, a field-programmable gate array, or another suitable component. Generally, each processor 118A, 118B, 118C, 118D executes instructions and manipulates data for soil corrosion analysis. Each processor 118A, 118B, 118C, 118D executes a functionality required to simulate soil corrosion relative to soil properties and to monitor and correct field soil corrosion patterns.

Interfaces 116A, 116B, 116C, 116D are used by different components of the example system 100 for communicating with other component systems in a distributed environment—including within the example system 100—connected to the network 108. Generally, the interfaces 116A, 116B, 116C, 116D each include logic encoded in software and/or hardware in a suitable combination and operable to communicate with the network 108. More specifically, the interfaces 116A, 116B, 116C, 116D may each include software supporting one or more communication protocols associated with communications such that the network 108 or interface's hardware is operable to communicate physical signals within and outside of the illustrated system 100.

The memory 1114A, 114B, 114C, 114D may include any type of memory or database module and may take the form of volatile and/or non-volatile memory including, without limitation, magnetic media, optical media, random access memory, read-only memory, removable media, or any other suitable local or remote memory component. The memory 1114A, 114B, 114C, 114D may store various objects or data, including caches, classes, frameworks, applications, backup data, jobs, web pages, web page templates, database tables, database queries, repositories storing soil properties 122 and any other appropriate information including corrosion pattern models, and any corrosion control parameters, variables, algorithms, instructions, rules, constraints, or references thereto associated with the purposes of the server system 102, the computing device 104, the data collection system 106 and the corrosion control system 110, respectively.

There may be any number of computing devices 104 and data collection systems 106 associated with, or external to, the example system 100. Additionally, there may also be one or more additional client devices external to the illustrated portion of system 100 that are configured for interacting with the example system 100 via the network(s) 108. Further, the term “client,” “client device,” and “user” may be used interchangeably as appropriate without departing from the scope of the disclosure. Moreover, while client device may be described in terms of being used by a single user, the disclosure contemplates that many users may use one computer, or that one user may use multiple computers. As used in the present disclosure, the term “computer” is intended to encompass any suitable processing device. For example, although FIG. 1A illustrates a single server system 102, a single computing device 104, a single data collection system 106, a single network management system 110, the example system 100 can be implemented using a single, stand-alone computing device, two or more core systems 102, or multiple client devices. The server system 102, the computing device 104 and the corrosion control system 110 may include any computer or processing device such as, for example, a blade server, general-purpose personal computer, workstation, or any other suitable device. In other words, the present disclosure contemplates computers other than general purpose computers, as well as computers without conventional operating systems. Further, the server system 102 and the computing device 104 and the corrosion control system 110 may be adapted to execute any operating system or runtime environment. According to one implementation, the server system 102 may also include or be communicably coupled with an e-mail server, a Web server, a caching server, a streaming data server, and/or another suitable server, as described with reference to FIG. 1B.

FIG. 1B is a block diagram of a portion of the example system 100 that can be used to execute implementations of the present disclosure. In particular, FIG. 1B depicts a schematic diagram illustrating an example portion 101 of a variation of the example system 100 described with reference to FIG. 1A, in accordance with some example embodiments. The example portion 101 of the example system 100 illustrated in FIG. 1B includes the data collection system 106, a corrosion detection and classification system 120A, an action plan engine 120B, and a corrosion control system 110.

The data collection system 106 can include an autoclave chamber 128 that includes a soil sample 134 in a Miller box 136, in contact with a four-way ER probe 138, a potentiostat 140, a data logger 142, a thermocouple 144, reference electrodes 146, a force extortion system 148, counter electrodes 150, pH/oxidation-reduction potential (ORP) probes 152, and a water spray system 154. The data collection system 106 with the described components is designed to monitor and analyze various parameters in a controlled environment, such as an autoclave chamber. The autoclave chamber 128 provides a controlled environment for conducting experiments, by applying high pressure and variable temperature conditions to the sample 134.

The soil sample 134 is placed in the Miller box 136 to study the sample properties and reactions under test conditions to identify soil corrosion rate. The four-way ER probe 138 measures the electrical resistivity of the soil sample 134, which helps in understanding its moisture content, salinity, and other properties. The potentiostat 140 controls the voltage applied to the soil sample and measures the resulting current, which is essential for electrochemical experiments. The data logger 142 records data from various sensors and instruments over time, facilitating continuous monitoring and analysis. The thermocouple 144 measures the temperature within the autoclave chamber 128, ensuring that the experimental conditions are accurately maintained.

The reference electrodes 146 provide a stable reference potential for electrochemical measurements, providing accurate and consistent readings of anodic or cathodic conditions. The force extortion system 148 applies a controlled mechanical force to the soil sample 134, simulating real-world conditions for studying the response of the sample 134. The counter electrodes 150 work in conjunction with the reference electrodes and potentiostat to complete the electrochemical cell and facilitate soil corrosion measurements. The pH/ORP probes 152 measure the pH and ORP of the soil sample 134, providing insights into the chemical properties of the soil sample 134.

The water spray system 154 simulates rainfall or irrigation by spraying water onto the soil sample 134, facilitating for the study of the soil corrosion in response to multiple hydrological conditions. The data collection system 106 can provide a comprehensive analysis of the soil corrosion relative to the soil sample's physical, chemical, and electrochemical properties under controlled conditions. The data collection system 106 can transmit the collected data to the detection and classification system 120A that can process the collected data and generate an output for the corrosion control system 110.

The corrosion detection and classification system 120A includes a data processing engine 156 that identifies the corrosion patterns relative to the tested parameters and electrochemical conditions. The corrosion detection and classification system 120A analyzes the detected corrosion to classify its type (e.g., pitting, crevice, uniform) and severity. This helps in understanding the nature and extent of the corrosion problem.

The action plan engine 120B evaluates the data collected and the classification results to determine the best course of action. The action plan engine 120B generates recommendations for mitigating or preventing further corrosion based on the analysis. The recommended action plans can include maintenance schedules, material replacements, or protective coatings. The action plan engine 120B can automate a particular set of actions, such as triggering alarms, scheduling maintenance tasks, or adjusting environmental controls to mitigate corrosion of soli within designated areas, such as industrial pipelines and cables to protect the buried assets. The action plan engine 120B includes an automatic risk assessment system and an alert system. The automatic risk assessment system can process the soil corrosion and generated measurements to identify a potential damage to an exposed asset. The action plan engine 120B can determine an immediate and a long-term risk associated to a respective asset, the risk level depending on the potential damage to the exposed asset. The action plan engine 120B can include a GUI (e.g., GUI 126 described with reference to FIG. 1A) to generate displays indicating the identified risk. The action plan engine 120B can generate a trigger to send to the corrosion control system 110 (including a machine or a device) to perform a remedial action (e.g., prevent soil corrosion and protect associated asset by applying protection layers, prepare a site, rebury the exposed asset, backfill the trench, and install reference markers).

The corrosion control system 110 executes the recommended actions to control and prevent corrosion. The corrosion prevention actions can include applying and regulating cathodic protection by adjusting current intensity generated by a potentiostat (e.g., by using an operational amplifier for adjusting the potential of the counter electrode, which causes current adjustment) of the cathodic protection controller 112, based on the soil corrosivity. In some implementations, the corrosion prevention actions can include applying protective coatings, adjusting environmental conditions (e.g., adjusting soil humidity by activating water sprinklers), or performing maintenance tasks. The corrosion control system 110 provides feedback to the data collection system 106 to ensure that the implemented actions are effective and to make any necessary adjustments to subsequent tests. The corrosion control system 110 uses the feedback and new data to continuously improve the corrosion control strategies and ensure long-term protection to mitigate corrosion of soli within designated areas, such as industrial pipelines and cables to protect the buried assets.

The described example system 100, 101 provides a soil corrosivity simulator that facilitates corrosion analysis integrating various soil parameters when assessing corrosion rates. The described example system 100, 101 can simulate the complex interaction of all soil parameters simultaneously, providing a more comprehensive understanding of the corrosion mechanism. The soil corrosivity simulator facilitates modelling of soil corrosion under various conditions, integrating different factors-resistivity, moisture content, pH, ORP, packing density, temperature, and pressure. The described example system 100, 101 enables the observation and measurement of the combined effects of these parameters on corrosion rates. The described example system 100, 101 can give an accurate prediction of corrosion based on the soil condition in accelerated manner. The described example system 100, 101 considers various parameters simultaneously including the impact surface polarization by cathodic protection which is crucial to measure the effectiveness of cathodic protection system to control soil corrosion. The described example system 100, 101 system can be optimized to enhance corrosion prevention measures for buried assets in various field operations.

While portions of the example system 100 illustrated in FIGS. 1A and 1B are shown as individual modules that implement the various features and functionality through various objects, methods, or other processes, the hardware components can execute software that can include multiple sub-modules, third-party services, components, libraries, and such, as appropriate. Conversely, the features and functionality of various components can be combined into single components as appropriate.

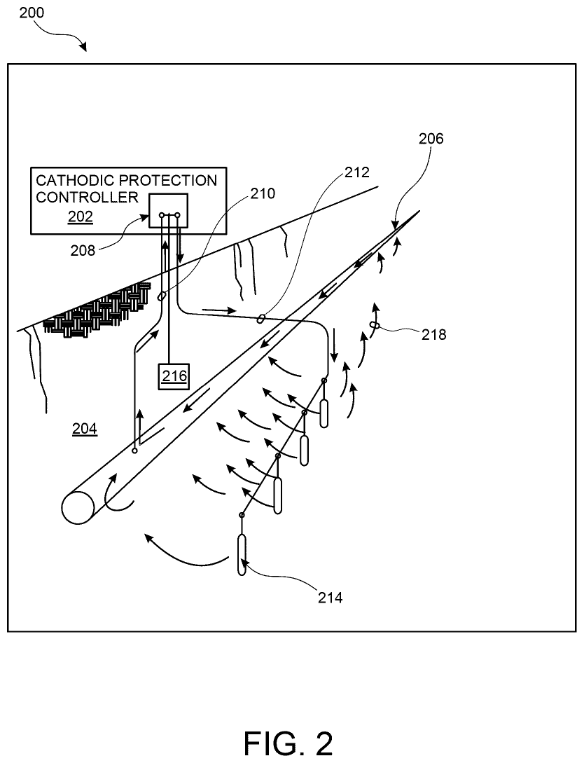

FIG. 2 illustrates an example soil corrosion protection system 200, according to some implementations of the present disclosure. The example soil corrosion protection system 200 can include the corrosion control system 110, described with reference to FIGS. 1A and 1B. The example soil corrosion protection system 200 can include a cathodic protection controller 202 (e.g., the cathodic protection controller 112, described with reference to FIGS. 1A and 1B). The cathodic protection controller 202 can be installed in a region of interest 204. The region of interest 204 can be a soil environment including one or more buried assets 206, such as pipelines and/or cables.

The cathodic protection controller 202 can include an operational amplifier 208, a drain wire 210, an anode header cable 212, a galvanic anode (working electrode) 214, and a reference electrode 216. The cathodic protection controller 202 can control the current provided to the region of interest 204 by managing the potential difference between the galvanic anode 214 and the reference electrode 216. The cathodic protection controller 202 can adjust the potential between the galvanic anode 214 and the reference electrode 216 based on signals received from an action plan engine to prevent corrosion within the region of interest 204. For example, the cathodic protection controller 202 can include (humidity and temperature) sensors to monitor corrosion risk and can monitor the potential by using a potentiostat that continuously monitors the potential difference between the galvanic anode 214 and the reference electrode 216.

The cathodic protection controller 202 can adjust the potential difference between the galvanic anode 214 and the reference electrode 216. The adjustment controls the current flowing through the galvanic anode 214. The cathodic protection controller 202 can include a control mechanism, such as an operational amplifier 208. The cathodic protection controller 202 uses the operational amplifier 208 to adjust the potential of the region of interest 204.

The amplifier ensures that the potential difference between the galvanic anode 214 and the reference electrode 216 remains constant, by controlling the current 218. To increase the current, the cathodic protection controller 202 can increase the set potential difference. To decrease the current, the cathodic protection controller 202 can reduce the set potential difference. The cathodic protection controller 202 can periodically calibrate the potentiostat and reference electrodes to maintain accurate control and measurements. The drain wire 210 provides a path to ground for any stray currents or electrical noise. The cathodic protection controller 202 can facilitate quantification of corrosion related parameters of a region of interest 204 that can be processed to prevent damage due to the asset exposure due to soil corrosion.

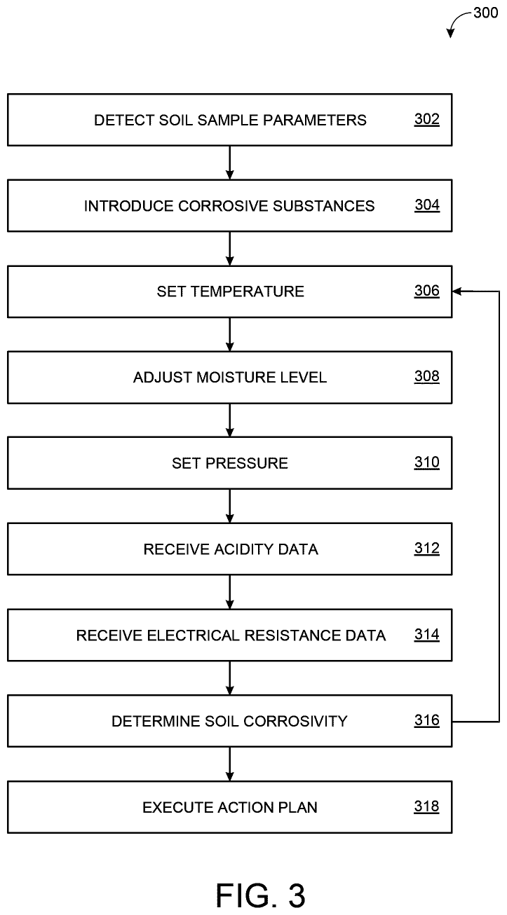

FIG. 3 is a flowchart illustrating an example process for cable exposure identification. Referring to FIGS. 1A, 1B, and 2, the process 300 can be performed by any components of the example systems 100, 101, and 200.

At 302, soil sample parameters of a region of interest are detected. The region of interest can include a soil environment surrounding a portion of an asset (e.g., a buried cable used for communication networks or pipeline used for gas or oil flow). The region of interest can be a rectangular area within a set of rectangular areas distributed along the length of the buried asset. The soil sample parameters can include a soil acidity (pH level), a soil humidity, a soil temperature, and other corrosion associated soil parameters.

At 304, the soil sample can be placed inside an autoclave chamber. The autoclave chamber can introduce corrosive agents like hydrogen sulfide, carbon dioxide, and other chemicals in order to replicate the field conditions that cause soil corrosion.

At 306, a temperature inside an autoclave chamber is set to simulate the soil corrosion relative to temperature changes. For an accurate monitoring of temperature, a thermocouple is inserted inside the autoclave chamber.

At 308, a moisture level inside the autoclave chamber is adjusted by activating a sprinkler system to simulate the soil corrosion relative to moisture conditions. For example, the soil resistivity box can include a water spray rod, including multiple nozzles, designed to evenly distribute water across the surface of the soil sample, at a set flow rate that can affect the injected water volume. The function of the water spray system is to simulate the pipelines exposure to moisture which can lead to corrosion.

At 310, a force exertion system is activated to apply a controlled pressure onto the soil sample at the set temperature and moisture level such that The effect of packing density of the soil can be assessed by incorporating a force exertion system. The force exertion system can apply a uniformly distributed pressure on a top surface of the soil sample. For example, the soil resistivity box can be closed with a movable lid that is connected to a force exertion system that can apply two equal loads to generate a controlled pressure onto the soil simulating the mechanical forces encountered in industrial environments. The force excretion system is intended to simulate the fluid flow pressure, accumulated weight of deposits, or other stress that can contribute to corrosion. The pressure testing can help determine the impact of pressure in the emergence and progression of soil corrosion as well as the effect of the packing density of the soil.

At 312, an acidity level and an ORP level of the soil are received from a pH/ORP probe inserted into or contacting the soil. The soil pH/ORP Probe can be positioned near the moistened soil surface for a real-time monitoring of the acidity or alkalinity as well as the oxidation-reduction potential (ORP) of the environment. The pH and ORP can significantly affect the rate of corrosion. In acidic environment (where pH is low), the corrosion rate tends to be high. A high ORP can indicate the presence of oxidizing agents, which makes the environment more susceptible to corrosion.

At 314, electrical resistance data of the soil is received, from a four-way electrical resistance probe. The electrical resistance measurements include placing non-conductive metal electrodes in a straight line on the soil surface, spaced equally part, with a depth not exceeding 5% of the minimum distance between the electrodes. Current is supplied through the outer electrodes, inducing the current flow, and the resulting voltage drop between the inner electrodes is measured. Low resistivity soils lead to high corrosion rates as they facilitate the flow of electrical currents, driving electrochemical reactions. High resistivity soil reduces the risk of soil corrosion. The four-way electrical resistance probe measures corrosion rate by determining the metal loss due to corrosion over time. The four-way electrical resistance probe includes four separate electrodes, facilitating the measurement of corrosion rates in different positions on the metal surface. The four-way design is essential in identifying areas where corrosion is more susceptible, facilitating for a better understanding of the corrosion mechanism. Prior to conducting corrosion measurements, the four-way electrical resistance probe can be connected to a potentiostat for polarization. An electrical resistance data logger can be coupled to the electrical resistance probe to receive and record corrosion rate data

At 316, a soil corrosivity is determined by processing the electrical resistance data, the soil corrosivity defining a soil corrosion rate of the soil sample relative to the temperature and the moisture level at the controlled pressure. In some implementations, determining soil corrosivity includes processing the electrical resistance data and the acidity level of the soil using the following formula:

ρ=R A/L

Where ρ is resistivity (ohm.cm), R is the resistance (ohms), A is the outer electrodes' cross-sectional area (cm2), and L is separation distance between the inner electrodes (cm). Different positions and directions electrical resistance probe can lead to different corrosion behavior such as corrosion under deposit, localized corrosion, etc., due to different factors such as gravity effect, moisture condensation, and temperature variation.

At 318, an action plan defining an action to prevent soil corrosion is determined, by the one or more processors. The action plan can be identified based on the simulated soil corrosivity. For example, the simulated corrosivity trends compared to measurements from a region of interest can be used to trigger an initiation of the action plan, and a modification of an electrical potential for cathodic protection to protect buried industrial assets. The action plan is automatically executed by generating a trigger, by the one or more processors, to activate an operation of a cathodic protection controller and an industrial machine configured to perform a remedy operation (e.g., digging, recoating, and reburying operation).

The example process 300 facilitates optimization of corrosion prevention. One of the greatest benefits of corrosion monitoring and prevention is maintenance of safe and secure pipeline operations, avoiding accidents including oil leaks. Corrosion rates can vary widely depending on the pipeline's location, environment conditions, as well as soil properties and corrosivity. Soil corrosion is a critical challenge especially in buried metal structures including pipelines and storage tanks. The example process 300 facilitates accurate simulation of soil corrosion relative to various parameters including soil resistivity, pH, ORP, and moisture content. The example process 300 enables accurate simulations using soil samples having different soil properties to accurately predict and control corrosion, facilitating evaluation of these interdependent parameters.

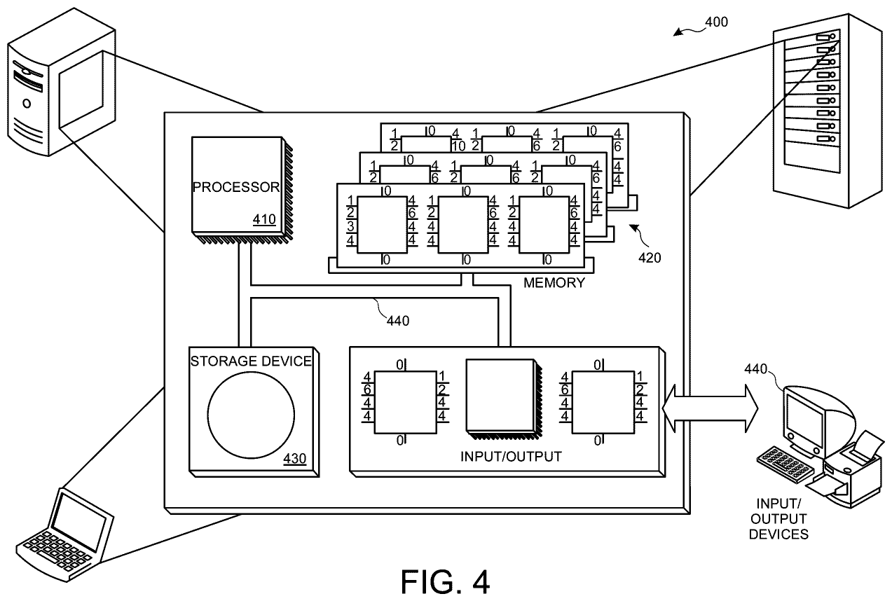

FIG. 4 depicts a block diagram illustrating a computing system 400, in accordance with some example embodiments. Referring to FIGS. 1A and 1B, the computing system 400 can be used to implement the server system 102 and/or any other components of the example system 100.

As shown in FIG. 4, the computing system 400 can include a processor 410, a memory 420, a storage device 430, and input/output devices 440. The processor 410, the memory 420, the storage device 430, and the input/output devices 440 can be interconnected using a system bus 450. The processor 410 is capable of processing instructions for execution within the computing system 400. Such executed instructions can implement one or more components of, for example, the example system 100. In some implementations of the current subject matter, the processor 410 can be a single-threaded processor. Alternately, the processor 410 can be a multi-threaded processor. The processor 410 is capable of processing instructions stored in the memory 420 and/or on the storage device 430 to display graphical information for a user interface provided using the input/output device 440.

The memory 420 is a computer readable medium such as volatile or non-volatile that stores information within the computing system 400. The memory 420 can store data structures representing configuration object databases, for example. The storage device 430 is capable of providing persistent storage for the computing system 400. The storage device 430 can be a floppy disk device, a hard disk device, an optical disk device, or a tape device, or other suitable persistent storage means. The input/output device 440 provides input/output operations for the computing system 400. In some implementations of the current subject matter, the input/output device 440 includes a keyboard and/or pointing device. In various implementations, the input/output device 440 includes a display unit for displaying graphical user interfaces.

According to some implementations of the current subject matter, the input/output device 440 can provide input/output operations for a network device. For example, the input/output device 440 can include Ethernet ports or other networking ports to communicate with one or more wired and/or wireless networks (e.g., a local area network (LAN), a wide area network (WAN), the Internet).

In some implementations of the current subject matter, the computing system 400 can be used to execute various interactive computer software applications that can be used for organization, analysis and/or storage of data in various (e.g., tabular) format (e.g., Microsoft Excel®, and/or any other type of software). Alternatively, the computing system 400 can be used to execute any type of software applications. These applications can be used to perform various functionalities, e.g., planning functionalities (e.g., generating, managing, editing of spreadsheet documents, word processing documents, and/or any other objects), computing functionalities, or communications functionalities. The applications can include various add-in functionalities or can be standalone computing products and/or functionalities. Upon activation within the applications, the functionalities can be used to generate the user interface provided using the input/output device 440. The user interface can be generated and presented to a user by the computing system 400 (e.g., on a computer screen monitor).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system can include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random-access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user can provide input to the computer. Other kinds of devices can be used to provide for interaction with a user as well. For example, feedback provided to the user can be any form of sensory feedback, such as for example visual feedback, auditory feedback, or tactile feedback; and input from the user can be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

The preceding figures and accompanying description illustrate example processes and computer implementable techniques. The environments and systems described above (or their software or other components) may contemplate using, implementing, or executing any suitable technique for performing these and other tasks. It can be understood that these processes are for illustration purposes only and that the described or similar techniques may be performed at any appropriate time, including concurrently, individually, in parallel, and/or in combination. In addition, many of the operations in these processes may take place simultaneously, concurrently, in parallel, and/or in different orders than as shown. Moreover, processes may have additional operations, fewer operations, and/or different operations, so long as the methods remain appropriate.

In other words, although the disclosure has been described in terms of certain implementations and generally associated methods, alterations and permutations of these implementations, and methods will be apparent to those skilled in the art. Accordingly, the above description of example implementations does not define or constrain the disclosure. Other changes, substitutions, and alterations are also possible without departing from the spirit and scope of the disclosure.

A number of implementations of the present disclosure have been described. Nevertheless, it will be understood that various modifications may be made without departing from the spirit and scope of the present disclosure. Accordingly, other implementations are within the scope of the following claims.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application.

Example 1. A computer-implemented method comprising: setting a temperature inside an autoclave chamber to simulate corrosion conditions of a soil sample extracted from a region of interest comprising buried pipelines; adjusting a moisture level inside the autoclave chamber to simulate moisture corrosion conditions; activating a force exertion system to apply a controlled pressure onto the soil sample at the moisture level; receiving, from a four-way electrical resistance probe, electrical resistance data of the soil; determining a soil corrosivity by processing the electrical resistance data, the soil corrosivity defining a soil corrosion rate of the soil sample relative to the temperature and the moisture level at the controlled pressure; and adjusting a potential to minimize the soil corrosion rate in the region of interest to protect the buried pipelines.

Example 2. The computer-implemented method of the previous example, wherein the four-way electrical resistance probe comprises four separate electrodes, a potentiostat, and an electrical resistance data logger.

Example 3. The computer-implemented method of any of the previous examples, wherein the four separate electrodes are placed in a substantially straight line on a surface of the soil.

Example 4. The computer-implemented method of any of the previous examples, further comprising: adjusting a polarization of the potentiostat connected to the electrical resistance data logger for potential control.

Example 5. The computer-implemented method of any of the previous examples, wherein determining the soil corrosivity comprises measuring a metal loss and adjusting the potential to minimize the soil corrosion comprises an adjustment of a cathodic protection.

Example 6. The computer-implemented method of any of the previous examples, further comprising: receiving, from a pH probe inserted into or contacting the soil, an acidity level of the soil, wherein determining soil corrosivity comprises processing the electrical resistance data and the acidity level of the soil.

Example 7. The computer-implemented method of any of the previous examples, further comprising: triggering an introduction of corrosive substances comprising hydrogen sulfide, and carbon dioxide to replicate conditions causing the soil corrosivity.

Example 8. A computer-implemented system comprising: memory storing application programming interface (API) information; and a server performing operations comprising: setting a temperature inside an autoclave chamber to simulate corrosion conditions of a soil sample extracted from a region of interest comprising buried pipelines; adjusting a moisture level inside the autoclave chamber to simulate moisture corrosion conditions; activating a force exertion system to apply a controlled pressure onto the soil sample at the moisture level; receiving, from a four-way electrical resistance probe, electrical resistance data of the soil; determining a soil corrosivity by processing the electrical resistance data, the soil corrosivity defining a soil corrosion rate of the soil sample relative to the temperature and the moisture level at the controlled pressure; and adjusting a potential to minimize the soil corrosion rate in the region of interest to protect the buried pipelines.

Example 9. The computer-implemented system of the previous example, wherein the four-way electrical resistance probe comprises four separate electrodes, a potentiostat, and an electrical resistance data logger.

Example 10. The computer-implemented system of any of the previous examples, wherein the four separate electrodes are placed in a substantially straight line on a surface of the soil.

Example 11. The computer-implemented system of any of the previous examples, wherein the operations comprise: adjusting a polarization of the potentiostat connected to the electrical resistance data logger for potential control.

Example 12. The computer-implemented system of any of the previous examples, wherein determining the soil corrosivity comprises measuring a metal loss and adjusting the potential to minimize the soil corrosion comprises an adjustment of a cathodic protection.

Example 13. The computer-implemented system of any of the previous examples, wherein the operations comprise: receiving, from a pH probe inserted into or contacting the soil, an acidity level of the soil, wherein determining soil corrosivity comprises processing the electrical resistance data and the acidity level of the soil.

Example 14. The computer-implemented system of any of the previous examples, wherein the operations comprise: triggering an introduction of corrosive substances comprising hydrogen sulfide, and carbon dioxide to replicate conditions causing the soil corrosivity.

Example 15. A non-transitory computer-readable media encoded with a computer program, the computer program comprising instructions that when executed by one or more computers cause the one or more computers to perform operations comprising: setting a temperature inside an autoclave chamber to simulate corrosion conditions of a soil sample extracted from a region of interest comprising buried pipelines; adjusting a moisture level inside the autoclave chamber to simulate moisture corrosion conditions; activating a force exertion system to apply a controlled pressure onto the soil sample at the moisture level; receiving, from a four-way electrical resistance probe, electrical resistance data of the soil; determining a soil corrosivity by processing the electrical resistance data, the soil corrosivity defining a soil corrosion rate of the soil sample relative to the temperature and the moisture level at the controlled pressure; and adjusting a potential to minimize the soil corrosion rate in the region of interest to protect the buried pipelines.

Example 16. The non-transitory computer-readable media of the previous example, wherein the four-way electrical resistance probe comprises four separate electrodes, a potentiostat, and an electrical resistance data logger.

Example 17. The non-transitory computer-readable media of any of the previous examples, wherein the four separate electrodes are placed in a substantially straight line on a surface of the soil.

Example 18. The non-transitory computer-readable media of any of the previous examples, wherein the operations comprise: adjusting a polarization of the potentiostat connected to the electrical resistance data logger for potential control.

Example 19. The non-transitory computer-readable media of any of the previous examples, wherein determining the soil corrosivity comprises measuring a metal loss and adjusting the potential to minimize the soil corrosion comprises an adjustment of a cathodic protection.

Example 20. The non-transitory computer-readable media of any of the previous examples, wherein the operations comprise: receiving, from a pH probe inserted into or contacting the soil, an acidity level of the soil, wherein determining soil corrosivity comprises processing the electrical resistance data and the acidity level of the soil.

What is claimed is:

1. A computer-implemented method comprising:
setting a temperature inside an autoclave chamber to simulate corrosion conditions of a soil sample extracted from a region of interest comprising buried pipelines;
adjusting a moisture level inside the autoclave chamber to simulate moisture corrosion conditions;
activating a force exertion system to apply a controlled pressure onto the soil sample at the moisture level;
receiving, from a four-way electrical resistance probe, electrical resistance data of the soil;
determining a soil corrosivity by processing the electrical resistance data, the soil corrosivity defining a soil corrosion rate of the soil sample relative to the temperature and the moisture level at the controlled pressure; and
adjusting a potential to minimize the soil corrosion rate in the region of interest to protect the buried pipelines.

2. The computer-implemented method of claim 1, wherein the four-way electrical resistance probe comprises four separate electrodes, a potentiostat, and an electrical resistance data logger.

3. The computer-implemented method of claim 2, wherein the four separate electrodes are placed in a substantially straight line on a surface of the soil.

4. The computer-implemented method of claim 2, further comprising:
adjusting a polarization of the potentiostat connected to the electrical resistance data logger for potential control.

5. The computer-implemented method of claim 1, wherein determining the soil corrosivity comprises measuring a metal loss and adjusting the potential to minimize the soil corrosion comprises an adjustment of a cathodic protection.

6. The computer-implemented method of claim 1, further comprising:
receiving, from a pH probe inserted into or contacting the soil, an acidity level of the soil, wherein determining soil corrosivity comprises processing the electrical resistance data and the acidity level of the soil.

7. The computer-implemented method of claim 1, further comprising:
triggering an introduction of corrosive substances comprising hydrogen sulfide, and carbon dioxide to replicate conditions causing the soil corrosivity.

8. A computer-implemented system comprising:
memory storing application programming interface (API) information; and
a server performing operations comprising:
setting a temperature inside an autoclave chamber to simulate corrosion conditions of a soil sample extracted from a region of interest comprising buried pipelines;
adjusting a moisture level inside the autoclave chamber to simulate moisture corrosion conditions;
activating a force exertion system to apply a controlled pressure onto the soil sample at the moisture level;
receiving, from a four-way electrical resistance probe, electrical resistance data of the soil;
determining a soil corrosivity by processing the electrical resistance data, the soil corrosivity defining a soil corrosion rate of the soil sample relative to the temperature and the moisture level at the controlled pressure; and
adjusting a potential to minimize the soil corrosion rate in the region of interest to protect the buried pipelines.

9. The computer-implemented system of claim 8, wherein the four-way electrical resistance probe comprises four separate electrodes, a potentiostat, and an electrical resistance data logger.

10. The computer-implemented system of claim 9, wherein the four separate electrodes are placed in a substantially straight line on a surface of the soil.

11. The computer-implemented system of claim 9, wherein the operations comprise:
adjusting a polarization of the potentiostat connected to the electrical resistance data logger for potential control.

12. The computer-implemented system of claim 8, wherein determining the soil corrosivity comprises measuring a metal loss and adjusting the potential to minimize the soil corrosion comprises an adjustment of a cathodic protection.

13. The computer-implemented system of claim 8, wherein the operations comprise:
receiving, from a pH probe inserted into or contacting the soil, an acidity level of the soil, wherein determining soil corrosivity comprises processing the electrical resistance data and the acidity level of the soil.

14. The computer-implemented system of claim 8, wherein the operations comprise:
triggering an introduction of corrosive substances comprising hydrogen sulfide, and carbon dioxide to replicate conditions causing the soil corrosivity.

15. A non-transitory computer-readable media encoded with a computer program, the computer program comprising instructions that when executed by one or more computers cause the one or more computers to perform operations comprising:
setting a temperature inside an autoclave chamber to simulate corrosion conditions of a soil sample extracted from a region of interest comprising buried pipelines;
adjusting a moisture level inside the autoclave chamber to simulate moisture corrosion conditions;
activating a force exertion system to apply a controlled pressure onto the soil sample at the moisture level;
receiving, from a four-way electrical resistance probe, electrical resistance data of the soil;
determining a soil corrosivity by processing the electrical resistance data, the soil corrosivity defining a soil corrosion rate of the soil sample relative to the temperature and the moisture level at the controlled pressure; and
adjusting a potential to minimize the soil corrosion rate in the region of interest to protect the buried pipelines.

16. The non-transitory computer-readable media of claim 15, wherein the four-way electrical resistance probe comprises four separate electrodes, a potentiostat, and an electrical resistance data logger.

17. The non-transitory computer-readable media of claim 16, wherein the four separate electrodes are placed in a substantially straight line on a surface of the soil.

18. The non-transitory computer-readable media of claim 16, wherein the operations comprise:
adjusting a polarization of the potentiostat connected to the electrical resistance data logger for potential control.

19. The non-transitory computer-readable media of claim 15, wherein determining the soil corrosivity comprises measuring a metal loss and adjusting the potential to minimize the soil corrosion comprises an adjustment of a cathodic protection.

20. The non-transitory computer-readable media of claim 15, wherein the operations comprise:
receiving, from a pH probe inserted into or contacting the soil, an acidity level of the soil, wherein determining soil corrosivity comprises processing the electrical resistance data and the acidity level of the soil.
